SEQUENCE LISTING (13 sequences)

SEQ ID NO: 1 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 2 - DNA, 4107 bp, Homo sapiens
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 3 - protein, 11 aa, Artificial Sequence
  Synthetic acetylated peptide
  MOD_RES (6)..(6): ACETYLATION
KKKATKKAAGA

SEQ ID NO: 4 - DNA, 23 bp, Artificial Sequence
  Synthetic oligonucleotide
ggcgaattcgaacacgcagatgc

SEQ ID NO: 5 - DNA, 24 bp, Artificial Sequence
  Synthetic oligonucleotide
cttccagcggatagaatggcgccg

SEQ ID NO: 6 - DNA, 29 bp, Artificial Sequence
  Synthetic oligonucleotide
gcactgatcatgaactcctctggatctac

SEQ ID NO: 7 - DNA, 28 bp, Artificial Sequence
  Synthetic oligonucleotide
gagaatagggttggcaccagcagcgcac

SEQ ID NO: 8 - DNA, 20 bp, Artificial Sequence
  Synthetic oligonucleotide
cgacgcaggtgtcgcaggtc

SEQ ID NO: 9 - DNA, 19 bp, Artificial Sequence
  Synthetic oligonucleotide
cacaactcctccgcgcaac

SEQ ID NO: 10 - DNA, 17 bp, Artificial Sequence
  Synthetic oligonucleotide
cgtcttcaccatggaga

SEQ ID NO: 11 - DNA, 21 bp, Artificial Sequence
  Synthetic oligonucleotide
cggccatcacgcccacagttt

SEQ ID NO: 12 - DNA, 24 bp, Artificial Sequence
  Synthetic oligonucleotide
ggctattcggctatgactgggcac

SEQ ID NO: 13 - DNA, 26 bp, Artificial Sequence
  Synthetic oligonucleotide
gagcaaggtgagatgacaggagatcc

(19) United States
(12) Patent Application Publication
(10) Pub. No.: US 2007/0065890 A1
(43) Pub. Date: Mar. 22, 2007

(54) Method for promoting formation of facultative heterochromatin

(76) Inventors: Danny F. Reinberg, Warren, NJ (US); Alejandro Vaquero, Barcelona (ES)

Correspondence Address: Jane Massey Licata;Licata & Tyrrell P.C., 66 E. Main Street, Marlton, NJ, 08053, US

(21) Appl. No.: 11/506,293

(22) Filed: Aug. 18, 2006

Related U.S. Application Data
(60) Provisional application No. 60/709,656, filed on Aug. 19, 2005.

(51) Int. Cl. G01N 33/574; C12P 21/08; C07K 16/30

(52) U.S. Cl. 435/7.23; 530/388.8; 435/455

(57) ABSTRACT

The present invention is a method for promoting facultative heterochromatin formation using SirT1. Using novel anti-SirT1 and anti-acetylated histone H1 antibodies, human SirT1 protein was shown to interact with and deacetylate histone H1 at lysine 26. Moreover, SirT1 was shown to mediate deactylation of histones H3 and H4 and spreading of hypomethylated H3-K79 with resultant silencing. Using the anti-SirT1 antibody, methods of diagnosing cancer are also provided.

### INTRODUCTION

This application claims benefit of U.S. Provisional Patent Application Ser. No. 60/709,656, filed Aug. 19, 2005, the contents of which are incorporated herein by reference in their entirety.

This invention was made in the course of research sponsored by the National Institutes of Health (NIH Grant No. GM 37120) and the National Cancer Institute (NCI Grant No. PA30 CA08748. The U.S. government may have certain rights in this invention.

### BACKGROUND OF THE INVENTION

Nuclear DNA is organized into chromatin which is composed of nucleosomes allowing proper compaction of the genetic information (Luger, et al. (1997) Nature 389:251-260). The N-terminal regions of the core histones protrude from the nucleosome and are subjected to a series of posttranslational modifications (Shiio and Eisenman (2003) Proc. Natl. Acad. Sci. USA 100:13225-13230; Vaquero, et al. (2003) Sci. Aging Knowledge Environ. 14:RE4). These modifications can alter the local properties of nucleosomes and also recruit factors that can further alter local chromatin structure (Berger (2002) Curr. Opin. Genet. Dev. 12:142-148).

Another level of chromatin organization in higher eukaryotes is the formation of the 30 nm fiber, which is apparently refractory to transcription and, in general, contains nucleosomal tails that are hypoacetylated, methylated, and include the linker histone H1 (Wolffe (1998) Chromatin: Structure and Function, Academic Press, San Diego, Calif. ; Morales, et al. (2001) Biochimie 83: 1029-1039). Most studies involving histone H1 have been performed in vitro. Only a few studies have determined a connection between histone H1 and other components of the chromatin machinery, and the functional relationship between the core histones and histone H1 in the establishment of higher order chromatin structure is under investigation (Carruthers, et al. (1998) Biochemistry 37:14776-14787; Hansen (2002) Annu. Rev. Biophys. Biomol. Struct. 31:361-392).

Another characteristic of higher order chromatin structure is its overall hypoacetylation of histones H3 and H4 (Braunstein, et al. (1993) Genes Dev. 7:592-604). Histone deacetylases (HDACs) are divided into three families based on homology with three different HDACs isolated from Saccharomyces cerevisiae: Rpd3p (class I), Hdalp (class II), and Sir2p (class III) (Gray and Ekstrom (2001) Exp. Cell Res. 262:75-83). Sir2p is an NAD⁺-dependent HDAC (Imai, et al. (2000) Nature 403:795-800; Landry, et al. (2000) Proc. Natl. Acad. Sci. USA 97:5807-5811) that participates in the formation of compacted chromatin in budding yeast. This compacted chromatin is hypoacetylated in histones H3 and H4, especially at H4-K16 (Braunstein, et al. (1996) Mol. Cell. Biol. 16:4349-4356), whose acetylated form is a mark for active genes (Johnson, et al. (1998) Nucleic Acids Res. 26:994-1001) and is involved in dosage compensation in D. melanogaster (Turner, et al. (1992) Cell 69:375-384). However, an in vivo H4-K16 deacetylase has yet to be described in humans.

A protein domain of approximately 250 amino acids that contains an NAD⁺-dependent deacetylase activity defines the Sir2 family of proteins whose members are conserved from Saccharomyces to humans (Frye (2000) Biochem. Biophys. Res. Commun. 273:793-798). In Saccharomyces cerevisiae, there are four proteins homologous to ySir2p, named Hst (Homologues of Sir Two) 1-4 (Gartenberg (2000) Curr. Opin. Microbiol. 3:132-137). Although some of these proteins can affect silencing to different extents, their function is in general not clear. In humans, there are at least seven Sir2-like proteins (SirT 1-7). The elucidation of the function and protein interactions of the human SirT proteins provide therapeutic targets for the treatment of disease and increasing life span.

### SUMMARY OF THE INVENTION

The present invention is a method for promoting formation of facultative heterochromatin. The method of the invention involves contacting a cell with human sirtuin type 1 thereby modulating the acetylation of histones and promoting formation of facultative heterochromatin.

The present invention also relates to antibodies. One embodiment embraces an isolated antibody raised against a C-terminal fragment of human sirtuin type 1. Another embodiment embraces an isolated antibody raised against residues 21-31 of histone Hlb, wherein lysine 26 of said histone H1b is acetylated.

The present invention further relates to a method for diagnosing a cancer. The diagnostic method of this invention involves contacting a sample with an antibody raised against a C-terminal fragment of human sirtuin type 1, detecting the level of human sirtuin type 1 protein in the sample and comparing said level with a control, wherein an elevated level of human sirtuin type 1 protein in the sample as compared to the control is indicative of cancer. A kit for carrying out this diagnostic method is also provided.

### DETAILED DESCRIPTION OF THE INVENTION

Using novel anti-SirT1 and anti-acetylated histone H1 antibodies, it has now been found that human SirT1 protein interacts with and deacetylates histone H1 at lysine 26 and promotes formation of facultative heterochromatin. Moreover, RNAi-mediated decreased expression of SirT1 in human cells causes hyperacetylation of H4-K16 and H3-K9 in vivo. Using an inducible system directing expression of SirT1 fused to the Gal4-DNA binding domain and a Gal4-reporter integrated in euchromatin, Gal4-SirT1 expression resulted in the deacetylation of H4-K16 and H3-K9, recruitment of H1 within the promoter vicinity, drastically reduced reporter expression, and loss of H3-K79 methylation, a mark restricting silenced chromatin. Accordingly, the present invention relates to anti-SirT1 and anti-acetylated histone H1 antibodies and a method for SirT1-mediated facultative heterochromatin formation that includes deacetylation of histone tails (e.g., H4-K16 and H3-K9), recruitment and deacetylation of histone H1, and spreading of hypomethylated H3-K79 with resultant silencing. Such formation of facultative heterochromatin formation is useful, e.g., in the analysis of the molecular events and physiological results associated with gene silencing as well as in increasing the life span of a cell or organism.

The findings presented herein indicate that SirT1 is involved in the formation of repressive chromatin. Unexpectedly, the connection of SirT1 to chromatin structure has not been directly analyzed previously. Some earlier reports, however, supported a function for SirT1 as a chromatin factor (Brachmann, et al. (1995) Genes Dev. 9:2888-2902; Derbyshire, et al. (1996) Yeast 12:631-640). In addition, the Drosophila Sir2 (dSir2), an ortholog of SirT1, participates in position effect variegation (Newman, et al. (2002) Genetics 162:1675-1685; Rosenberg and Parkhurst (2002) Cell 109:447-458), and the C. elegans ortholog of SirT1, Sir2.1, is involved in silencing repetitive transgenes (Jedrusik and Schulze (2003) Mol. Cell. Biol. 23:3681-3691), as well as in increased life span (Tissenbaum and Guarente (2001) Nature 410:227-230). Moreover, Hst1 is involved in regulation of middle sporulation genes during vegetative growth and early meiosis, through its recruitment by transcription factors resulting in gene repression (McCord, et al. (2003) Mol. Cell. Biol. 23:2009-2016; Sutton, et al. (2001) Mol. Cell. Biol. 21:3514-3522; Xie, et al. (1999) EMBO J. 18:6448-6454).

It has now been shown that SirT1-mediated formation of repressive chromatin is mediated through at least four SirT1-coordinated events. First, SirT1 preferentially catalyzes the deacetylation of H4-K16. Previous reports have shown that acetylated H4-K16 is a “chromatin mark” associated with euchromatin (Johnson, et al. (1998) supra) and that heterochromatin is hypoacetylated in K16 (Jeppesen and Turner (1993) Cell 74:281-289). In yeast, although Sir2p can deacetylate different residues within the H3 and H4 tails, only H4-K16 deacetylation seems to be Sir2p specific (Braunstein, et al. (1993) supra) . In fact, Sir2 function is controlled by Sas2, the H4-K16 HAT that together with Sir2 create a gradient of H4-K16 acetylation in yeast that restricts silencing to specific regions (Suka, et al. (2002) Nat. Genet. 32:378-383).

Second, SirT1 interacts with histone H1, and this interaction recruits histone H1 to establish repressive chromatin. In this regard, C. elegans histone H1.1 is involved in development (Jedrusik and Schulze (2001) Development 128:1069-1080), can affect telomeric position effect when introduced in S. cerevisiae, and participates with the Sir2 homolog, Sir-2.1, in the silencing of repetitive transgenes in C. elegans (Jedrusik and Schulze (2003) supra). In addition, in the filamentous fungus Ascobulus immersus, histone H1 is essential for increased life span. In mammals, H1 is essential for development, as is mouse SIR2α (SirT1) (Fan, et al. (2003) i Mol. Cell. Biol. 23:4559-4572). Moreover, H1 has been found to be recruited to the MyoD gene by the transcription repressor Msx-1 involved in muscle differentiation (Lee, et al. (2004) Science 304:1675-1678).

Third, SirT1 catalyzes deacetylation of H1. This is the first evidence that H1 is acetylated at a lysine residue, K26, which is targeted for deacetylation by SirT1. It was found that H1-K26 is acetylated in vivo and that SirT1 deacetylates acetyl H1b lysine 26 in vitro. The primary effect of core histone acetylation is believed to be the partial neutralization of the highly charged tails, which in turn weakens its interaction with DNA (Hong, et al. (1993) J. Biol. Chem. 268:305-314; Workman and Kingston (1998) Annu. Rev. Biochem. 67:545-579). In accordance with this, it is posited that since H1 is involved in formation of compacted chromatin structures in part by its interaction with linker DNA (Hansen (2002) supra), its deacetylation produced a similar effect as that shown for core histones. Thus, deacetylation of both core histones and H1 could produce a stronger effect on chromatin compaction than either separately.

Fourth, SirT1 targeted to a reporter integrated into euchromatic regions of the mammalian genome resulted in the reduction of methylated histone H3-K79 at the promoter and within the coding region. H3-K79 methylation restricts the spreading of Sir2p in yeast, and thus, methylation of this residue may constitute a “boundary mark” separating heterochromatic from euchromatic regions (Lacoste, et al. (2002) J. Biol. Chem. 277:30421-30424; Ng, et al. (2002) Genes Dev. 16:1518-1527; Ng, et al. (2002) Genes Dev. 16:1518-1527). This is consistent with the presence of other heterochromatin features which were observed, such as H3-K9 and H4-K20 methylation after SirT1 targeting to the reporter.

Not wishing to be bound by theory, it is believed the SirT1-coordinated events may include the recruitment of a factor that exchanges the H3/H4 tetramer or a specific histone demethylase with specificity for H3-K79. Previous studies have demonstrated that SirT1 interacts with some transcription factors (Luo, et al. (2001) Cell 107:137-148; Vaziri, et al. (2001) Cell 107:149-159; Muth, et al. (2001) EMBO J. 20:1353-1362; Fulco, et al. (2003) Mol. Cell 12:51-62; Senawong, et al. (2003) J. Biol. Chem. 278:43041-43050; Takata and Ishikawa (2003) Biochem. Biophys. Res. Commun. 301:250-257); and these factors likely recruit SirT1 to specific genes to repress transcription. Thus far, SirT1 has only been detected at the promoters of the MHC, myogenin (Fulco, et al. (2003) supra), aP2, and PPARγ (Picard, et al. (2004) Nature 429:771-776) genes. However, the complexity of the regulation of these promoters, wherein multiple transcription factors function simultaneously, complicates the analysis of SirT1 function on chromatin. This was circumvented herein by directing Gal4-SirT1 to a specific gene integrated in euchromatin so that the effects of SirT1 recruitment could be analyzed in detail.

Given the novel histone deacetylase function identified for human SirT1, the present invention is a method for promoting facultative heterochromatin formation in a cell by contacting the cell with human SirT1. As used in the context of the present invention, facultative heterochromatin is chromosomal material which is genetically inactive but has the faculty to return to the normal euchromatic state. Facultative heterochromatin formation in accordance with the particular embodiments f the present invention can be carried out in vitro, ex vivo, or in vivo in any eukaryotic cell, particularly human cells.

Human SirT1 protein, i.e., sirtuin type 1, is known in the art under GENBANK Accession No. NP_{—}036370 and provided herein as all or part of the amino acid sequence as provided in SEQ ID NO:1. As used in the context of the present invention, a fragment of human SirT1 protein includes the N-terminal amino acid residues of SirT1, namely amino acid residues 1-268 of SEQ ID NO:1; or the C-terminal amino acid residues of human SirT1 protein, namely amino acid residues 581-630 of SEQ ID NO:1.

Human SirT1, or a fragment thereof, can be purified from its natural source, recombinantly produced or chemically synthesized. Methods for recombinant production of human SirT1 or fragments thereof for use in the method of the present invention are well-known to those skilled in the art. Nucleic acids encoding human SirT1 or fragments thereof are known in the art and can readily be obtained from databases such as EMBL and GENBANK (see, e.g., accession number NM_{—}012238). The nucleotide sequence encoding human SirT1 is provided as SEQ ID NO:2. Nucleic acids encoding human SirT1 can be obtained by any method known in the art, e.g., by PCR amplification using synthetic primers hybridizable to the 3′ and 5′ ends of each sequence, and/or by cloning from a cDNA or genomic library using an oligonucleotide specific for each nucleotide sequence.

An encoded human SirT1 protein, which is depicted as SEQ ID NO:1 can be obtained by methods well-known in the art for protein purification and recombinant protein expression. For recombinant expression of human SirT1, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals can also be supplied by the native promoter of the human SirT1 gene, and/or its flanking regions.

A variety of host-vector systems can be utilized to express the protein coding sequence. These include, but are not limited to, mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements can be used.

Any method available in the art can be used for the insertion of DNA fragments into a vector to construct expression vectors containing a chimeric gene consisting of appropriate transcriptional/translational control signals and protein coding sequences. These methods can include in vitro recombinant DNA and synthetic techniques and in vivo recombinant techniques (genetic recombination). Expression of nucleic acid sequences encoding human SirT1, or a fragment thereof, can be regulated by a second nucleic acid sequence so that the gene or fragment thereof is expressed in a host transformed with the recombinant DNA molecule(s). For example, expression of the protein can be controlled by any promoter/enhancer known in the art. Further, the promoter may not be native to the gene for human SirT1. Promoters that can be used include, but are not limited to, the SV40 early promoter (Bernoist and Chambon (1981) Nature 290:304-310), the promoter contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. USA 78:1441-1445), the regulatory sequences of the metallothionein gene (Brinster et al. (1982) Nature 296:39-42); prokaryotic expression vectors such as the β-lactamase promoter (Villa-Kamaroff et al. (1978) Proc. Natl. Acad. Sci. USA 75:3727-3731) or the tac promoter (DeBoer et al. (1983) Proc. Natl. Acad. Sci. USA 80:21-25; Gilbert et al. (1980) Scientific American 242:79-94); plant expression vectors comprising the nopaline synthetase promoter (Herrar-Estrella et al. (1984) Nature 303:209-213) or the cauliflower mosaic virus 35S RNA promoter (Garder et al. (1981) Nucleic Acids Res. 9:2871), and the promoter of the photosynthetic enzyme ribulose bisphosphate carboxylase (Herrera-Estrella et al. (1984) Nature 310:115-120); promoter elements from yeast and other fungi such as the Gal4 promoter (Johnston et al. (1987) Microbiol. Rev. 51:458-476), the alcohol dehydrogenase promoter (Schibler et al. (1987) Ann. Rev. Genet. 21:237-257), the phosphoglycerol kinase promoter (Struhl et al. (1995) Ann. Rev. Genet. 29:651-674-257; Guarente (1987) Ann. Rev. Genet. 21:425-452), the alkaline phosphatase promoter (Struhl et al. (1995) Ann. Rev. Genet. 29:651-674-257; Guarente (1987) Ann. Rev. Genet. 21:425-452), and animal transcriptional control regions that exhibit tissue specificity and have been utilized in transgenic animals include alpha-myosin heavy chain promoter (Sanbe et al. (2003) Circ. Res. 92(6):609-16) or alpha-MHC (Kirchhefer, et al. (2003) Cardiovasc. Res. 59(2):369-79).

In general, a vector is used that contains a promoter operably linked to the nucleic acid sequence encoding human SirT1, or a fragment thereof, one or more origins of replication, and optionally, one or more selectable markers (e.g., an antibiotic resistance gene).

An expression vector containing the coding sequence, or a portion thereof, of human SirT1 can be made by subcloning the gene sequence into the EcoRI restriction site of each of the three pGEX vectors (glutathione S-transferase expression vectors; Smith and Johnson (1988) Gene 7:31-40). This allows for the expression of products in the correct reading frame.

Once recombinant human SirT1 expression vectors are identified and isolated, several methods known in the art can be used to propagate them. Using a suitable host system and growth conditions, recombinant expression vectors can be propagated and amplified in quantity. Expression vectors or derivatives which can be used include, but are not limited to, human or animal viruses such as vaccinia virus or adenovirus; insect viruses such as baculovirus, yeast vectors; bacteriophage vectors such as lambda phage; and plasmid and cosmid vectors.

In addition, a host cell strain can be chosen that modulates the expression of the inserted sequences, or modifies or processes the expressed proteins in the specific fashion desired. Expression from certain promoters can be elevated in the presence of certain inducers; thus expression of the genetically-engineered human SirT1 can be controlled. Furthermore, different host cells have characteristic and specific mechanisms for the translational and post-translational processing and modification (e.g., glycosylation, phosphorylation, etc.) of proteins. Appropriate cell lines or host systems can be chosen to ensure that the desired modification and processing of the foreign protein is achieved. For example, expression in a bacterial system can be used to produce an unglycosylated core protein, while expression in mammalian cells ensures native glycosylation of a heterologous protein. Furthermore, different vector/host expression systems can effect processing reactions to different extents.

A human SirT1 protein, or a fragment thereof, can be expressed as fusion or chimeric protein products containing the protein, or fragment, joined via a peptide bond to a heterologous protein sequence of a different protein. Such chimeric products can be made by ligating the appropriate nucleic acid sequences encoding the desired amino acids to each other by methods known in the art, in the proper coding frame, and expressing the chimeric products in a suitable host by methods commonly known in the art. Alternatively, such a chimeric product can be made by protein synthetic techniques, e.g., by use of a peptide synthesizer. Chimeric genes containing portions of human SirT1 fused to any heterologous protein-encoding sequences can be constructed.

Methods that can be used to carry out the foregoing are commonly known in the art. The cells used in the methods of this embodiment of the invention can either endogenously or recombinantly express human SirT1, or a fragment thereof. Recombinant expression of human SirT1 is carried out by introducing human SirT1 encoding nucleic acids into expression vectors and subsequently introducing the vectors into a cell to express human SirT1 or simply introducing human SirT1 encoding nucleic acids into a cell for expression using procedures well-known in the art (e.g., microinjection, liposome-mediated transfection, electroporation, or calcium phosphate precipitation). Expression can be from expression vectors or intrachromosomal, as is known in the art. See, e.g., Molecular Biology, Ausubel, F. M. et al. (eds.) Greene Publishing Associates, (1989), and other standard laboratory manuals.

Human SirT1 can be purified using any conventional protein purification method. Particular embodiments embrace affinity purification of human SirT1 using an antibody raised against a C-terminal fragment of human SirT1 and specifically recognizes human SirT1, but fails to react with the other members of the human Sirtuin family of proteins. Anti-human SirT1 antibodies, as well as other antibodies disclosed herein (i.e., anti-acetylated lysine 26 histone H1b), can be generated using methods that are well-known in the art and include, but are not be limited to, polyclonal, monoclonal, chimeric, single chain, Fab fragments, bispecific scFv fragments, Fd fragments and fragments produced by a Fab expression library.

For the production of antibodies, various hosts including goats, rabbits, rats, mice, humans, and others, may be immunized by injection with a protein or fragment thereof which has antigenic or immunogenic properties. Exemplary fragments for use in producing antibodies of the instant invention include the C-terminal portion of human SirT1 (i.e., amino acid residues 581-630 of SEQ ID NO:1) and amino acid residues 21-31 of histone H1b (i.e., SEQ ID NO:3) . Depending on the host species, various adjuvants can be used to increase the immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are particularly suitable.

An antibody of the invention can be generated by immunizing an animal with a peptide or fragment as disclosed herein. Generally, such peptides or fragments have an amino acid sequence consisting of at least five amino acids and more desirably at least 10 amino acids. Fragments can be generated by, for example, tryptic digestion and extraction from a preparative SDS-PAGE gel, by recombinant fragment expression and purification, or chemical synthesis. Further, such peptides or fragments can be fused with those of another protein such as keyhole limpet hemocyanin and antibody produced against the chimeric molecule.

Monoclonal antibodies of the invention can be prepared using any technique which provides for the production of antibody molecules by continuous cell lines in culture. These include, but are not limited to, the hybridoma technique, the human B-cell hybridoma technique, and the EBV-hybridoma technique (Kohler, et al. (1975) Nature 256:495-497; Kozbor, et al. (1985) J. Immunol. Methods 81:31-42; Cote, et al. (1983) Proc. Natl. Acad. Sci. 80:2026-2030; Cole, et al. (1984) Mol. Cell Biol. 62:109-120).

In addition, techniques developed for the production of humanized and chimeric antibodies, the splicing of mouse antibody genes to human antibody genes to obtain a molecule with appropriate antigen specificity and biological activity, can be used (Morrison, et al. (1984) Proc. Natl. Acad. Sci. 81, 6851-6855; Neuberger, et al. (1984) Nature 312:604-608; Takeda, et al. (1985) Nature 314:452-454). Alternatively, techniques described for the production of single chain antibodies can be adapted, using methods known in the art, to produce specific single chain antibodies. Antibodies with related specificity, but of distinct idiotypic composition, can be generated by chain shuffling from random combinatorial immunoglobulin libraries (Burton (1991) Proc. Natl. Acad. Sci. 88,11120-11123).

Antibodies can also be produced by inducing in vivo production in the lymphocyte population or by screening immunoglobulin libraries or panels of highly specific binding reagents as is well-known in the art (Orlandi, et al. (1989)Proc. Natl. Acad. Sci. 86: 3833-3837; Winter, et al. (1991) Nature 349:293-299).

Antibody fragments, which contain specific binding sites for the protein of interest can also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed to allow rapid and easy identification of monoclonal Fab fragments with the desired specificity (Huse, et al. (1989) Science 254:1275-1281).

Diabodies are also contemplated. A diabody refers to an engineered antibody construct prepared by isolating the binding domains (both heavy and light chain) of a binding antibody, and supplying a linking moiety which joins or operably links the heavy and light chains on the same polypeptide chain thereby preserving the binding function (see, Holliger et al. (1993) Proc. Natl. Acad. Sci. USA 90:6444; Poljak (1994) Structure 2:1121-1123). This forms, in essence, a radically abbreviated antibody, having only the variable domain necessary for binding the antigen. By using a linker that is too short to allow pairing between the two domains on the same chain, the domains are forced to pair with the complementary domains of another chain and create two antigen-binding sites. These dimeric antibody fragments, or diabodies, are bivalent and bispecific. It should be clear that any method to generate diabodies, as for example described by Holliger, et al. (1993) supra, Poljak (1994) supra, Zhu, et al. (1996) Biotechnology 14:192-196, and U.S. Pat. No. 6,492,123, herein incorporated by reference, can be used.

Various immunoassays can be used for screening to identify antibodies, or fragments thereof, having the desired specificity for human SirT1 or acetylated lysine 26 of histone H1b. Numerous protocols for competitive binding (e.g., ELISA), latex agglutination assays, immunoradiometric assays, and kinetics (e.g., BIACORE analysis) using either polyclonal or monoclonal antibodies, or fragments thereof, are well-known in the art. Such immunoassays typically involve the measurement of complex formation between a specific antibody and its cognate antigen. A two-site, monoclonal-based immunoassay utilizing monoclonal antibodies reactive to two non-interfering epitopes is suitable, but a competitive binding assay can also be employed. Such assays can also be used in the detection of, e.g., acetylated lysine 26 of histone H1b in a cell.

The anti-human SirT1 and anti-acetylated lysine 26 histone H1b antibodies find use in the purification of the respective SirT1 and acetylated histone H1b proteins as well as in the determination of efficacy and efficiency of the instant method. For example, using the anti-acetylated lysine 26 histone H1b antibody, it can be determined whether there is a decrease in the amount of acetylated histone H1b protein in a cell which has been contacted with human SirT1.

In accordance with the instant method, a cell is contacted with an effective amount of human SirT1 so that histone aceylation is modulated and facultative heterochromatin formation is promoted. Particular embodiments of the present invention embrace the deacetylation of histone H1b, H3 and H4 with the deacetylation of histones H2A, H1° and H1d (H1.2) also contemplated. Upon facultative heterochromatin formation, gene silencing commences. This is of particular use in providing proper control of chromatin structure, e.g., to modulate the effects of aging. Caloric restriction (and thus high levels of NAD⁺) is clearly related to increased life span in multiple organisms including mammals (Hasty (2001) Mech. Aging Dev. 122:1651-1662). Given the effect of SirT1 on chromatin, together with its requirement for NAD⁺, it is contemplated herein that SirT1 is useful in increasing life span. In this regard, it is of relevance that one of the chromatin elements that have been connected to aging is H1, since aging produces changes in the distribution of H1 subtypes (Lennox and Cohen (1983) J. Biol. Chem. 258:262-268; Parseghian, et al. (2000) Chromosome Res. 8:405-424; Parseghian, et al. (2001) J. Cell. Biochem. 83:643-659), deamination of H1 molecules (Lindner, et al. (1999) J. Cancer Res. Clin. Oncol. 125:182-186), and a loss of acetylation of serine 1 (Sarg, et al. (1999) Arch. Biochem. Biophys. 372:333-339).

Overexpression of SirT1 has been documented in cancer cells. For example, in an analysis of 225 samples (135 primary tumors, 47 cancer cell lines, and 43 normal tissues), differential expression of SirT1 in combination with CREBBP, HDAC7A, HDAC5 and PCAF could accurately distinguish between colorectal cancers and normal colorectal mucosa and the with SIRT1 and CREBBP, about 80% of independent breast tumor samples could be correctly classified. (Özda\check{g}, et al. (2006) BMC Genomics 7:90). Similarly, SIRT1 has been found to be consistently overexpressed (>2 fold) in acute myeloid leukemia samples compared with controls (Bradbury, et al. (2005) Leukemia 19(10):1751-9). SirT1 RNA and protein levels are also increased in drug-resistant cell lines compared with their drug-sensitive counterparts and also increased in biopsy tissue from cancer patients treated with chemotherapeutic agents (Chu, et al. (2005) Cancer Res. 65(22):10183-7). Thus, the present invention also relates to a method for diagnosing a cancer using the anti-SirT1 antibody of the present invention. Given that the anti-SirT1 antibody clearly detects SirT1 in the nuclei of human cells, this antibody is useful in diagnosing such cancers as colorectal cancer, breast cancer, acute myeloid leukemia, as well as drug-resistant cancer.

In general, an assay for diagnosing a cancer involves contacting a sample such as a biopsy sample, tissue, or cell from a subject having or suspected of having a cancer (e.g., colorectal, breast, or acute myeloid leukemia) and detecting the level of SirT1 protein in the sample as compared to a control. A subject having or suspected of having a cancer respectively includes an individual exhibiting symptoms indicative of cancer (e.g., a tumor) or having a family history of cancer which increases the risk for the development of cancer.

Detection of SirT1 protein levels in accordance with this diagnostic method is achieved using the instant anti-SirT1 antibody in quantitative or semi-quantitative immunoassays. Such immunoassays are well-known in the art and include, e.g., enzyme-linked immunosorbent, immunodiffusion chemiluminescent, immunofluorescent, immunohistochemical, radioimmunoassay, agglutination, complement fixation, immunoelectrophoresis, and immunoprecipitation assays and the like which can be performed in vitro, in vivo or in situ. Such standard techniques are well-known to those of skill in the art (see, e.g., “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984) J. Clin. Chem. Clin. Biochem. 22:895-904). Other well-known immunoassays include antibody capture assays, antigen capture assays, and two-antibody sandwich assays.

After detecting the level of SirT1 protein present in the sample, the results seen in a given sample can be compared with a control or standard. A control can be a healthy sample taken from the same subject, a statistically significant reference group of normal subjects or subjects that have cancer to provide diagnostic, prognostic, or predictive information pertaining the subject from whom the sample was obtained. When compared to a healthy sample or reference group of normal subjects, an elevated level of SirT1 is indicative of cancer in the subject from whom the sample was obtained. It is contemplated that the diagnostic method of the invention can be used alone or in combination with other well-known diagnostic or staging methods for cancer.

The present invention also provides a kit which is useful for carrying out the diagnostic method of the present invention. The kit generally includes a container which containing an anti-SirT1 antibody as disclosed herein. The kit also contains other solutions necessary or convenient for carrying out the invention. The container can be made of glass, plastic or foil and can be a vial, bottle, pouch, tube, bag, etc. The kit can also contain written information, such as procedures for carrying out the present invention; analytical information, such as the amount of reagent contained in the first container; or data describing the correlation between the level of SirT1 protein in a sample and the presence of a cancer. The container can further be in another container, e.g., a box or a bag, along with the written information.

It is contemplated that in addition to the anti-SirT1 antibody, the anti-H1b antibody disclosed herein will find application in diagnostic, prognostic, or predictive methods of cancer.

The invention is described in greater detail by the following non-limiting examples.

### EXAMPLE 1

Experimental Procedures

Antibodies. Monoclonal antibodies were generated using synthetically synthesized peptides to a C-terminal region of SirT1 corresponding to amino acids 581-630 (BiosChile, Chile). Antibody against SirT1 was purchased from ABCAM (Cambridge, Mass.). Histone antibodies were purchased from Cell Signaling (Beverly, Mass.) and Upstate Biotechnology, Inc. (Lake Placid, N.Y.). Antibodies against acetylated histones were purchased from Upstate Biotechnology, Inc. (Lake Placid, N.Y.), except acetylated H4 K16 (Serotec, Raleigh, N.C.) . Other antibodies used were histone trimethyl H3-K4 (ABCAM, Cambridge, Mass.), trimethyl H3-K9 (Sarma, et al. (2004) Methods Enzymol. 376:255-269), monomethyl H4-K20 (Upstate Biotechnology, Inc., Lake Placid, N.Y.), histone H1 (Upstate Biotechnology, Inc., Lake Placid, N.Y.), and Gal4-DBD (Upstate Biotechnology, Inc., Lake Placid, N.Y.). FLAG M2 and HA antibodies and antibody-conjugated agarose were purchased from SIGMA (St. Louis, Mo.) . Polyclonal antibodies against acetylated histone H1 lysine 26 were generated in rabbits using an acetylated (Ac) peptide (i.e., Lys-Lys-Lys-Ala-Thr-Lys^{Ac}-Lys-Ala-Ala-Gly-Ala; SEQ ID NO:3) as antigen. H1 antibodies were purified first with an unmodified peptide column followed by purification with the antigenic peptide column. The peptides corresponding to acetylated H4-K16 and acetylated H4-K12 were purchased from Global Peptide Services (Fort Collins, Colo.), and peptide corresponding to acetylated H3-K27 was purchased from ABCAM (Cambridge, Mass.) . Methylated H1 and unmodified peptides are described in the prior art (Sarma, et al. (2004) Methods Enzymol. 376:255-269).

In Vitro Binding Assays. Indicated recombinant proteins (1 μg) were incubated in buffer C (20 mM Tris-HCl [pH 7.9], 0.2 mM EDTA, 10% glycerol, 1 mM DTT, and 0.2 mM PMSF) containing 100 mM KCl and 0.01% NP40, at 4° C. overnight in the presence of anti-FLAG, anti-HA, or covalently cross-linked anti-SirT1 protein G. FLAG and HA beads were washed extensively with buffer C containing 100 mM KCl and 0.01% NP40, followed by washing with buffer C containing 500 mM KCl and 0.01% NP40. Proteins were eluted from FLAG and HA resins with FLAG and HA peptide, respectively (SIGMA, St. Louis, Mo.). SirT1 beads were washed with buffer C containing 300 mM KCl and 0.01% NP40, and proteins were eluted with 0.2 M glycine (pH 2.5). Bound and unbound proteins were visualized by western analysis.

In Vitro NAD⁺-Dependent Enzymatic Assays. Nicotinamide exchange reaction (NER) was performed accordingly to established methods (Landry, et al. (2000) supra). TLC plates were K6 silica gels 60 Å (WHATMAN, Florham Park, N.J.). Between 1.0 and 4.0 μpg of acetylated substrate was used in the NER assays. All reactions were incubated for 1 hour at 30° C. except in time course experiments. Between 0.1 and 2.0 nmol of enzyme were used in all assays as indicated. All in vitro assays used purified, recombinant SirT1, SirT2, or SirT3, as indicated. A final concentration of 5 mM NAD⁺ (SIGMA, St Louis, Mo.) was used in all NAD⁺-dependent assays. In deacetylation assays, 8 μg of core histones isolated from HeLa cells treated with TSA and sodium butyrate were used (Loyola, et al. (2001) Genes Dev. 15:2837-2851). Histone deacetylation was visualized by TAU electrophoresis followed by COOMASSIE blue staining using a modification of a published procedure (Lennox and Cohen (1989) Methods Enzymol. 170:532-549), and by standard western analysis using the specified antibodies. In the time course experiment, the reaction was scaled up and samples were taken at the indicated time points after addition of NAD⁺. Western analysis was performed using the indicated antibodies and signal intensity was determined using IMAGEQUANT software (MOLECULAR DYNAMICS, Sunnyvale, Calif.) . All points were normalized by the signal intensity at time 0 and represented as percentage.

Detection of H1 acetylation was performed by using 4 μg of histone H1 (Upstate Biotechnology, Inc., Lake Placid, N.Y.) as an acetylated substrate for SirT1 in the NER. Deacetylation was determined by first performing a deacetylation assay using 20 and 40 μg of H1 and 6 nmol of SirT1. H1 was then isolated in the supernatant of chilled 5% perchloric acid, TCA precipitated (Albig, et al. (1998) FEBS Lett. 435:245-250), and resuspended in buffer C containing 100 mM KCl. Deacetylated H1 was then used in a subsequent NER. In vitro acetylation of H1 or recombinant H1b expressed in E. coli was carried out by incubating 10 μg H1 with 2 nmol baculovirus-generated p300 in the presence of 0.875 μCi [H³]-Ac-CoA (Amersham, Piscataway, N.J.) in HAT buffer (75 mM Tris-HCl [pH 8.8], 135 mM NaCl). Deacetylation was carried out with the specified enzyme as above using 1 μg of the labeled Hi. Deacetylation was visualized by spotting on P81 paper (WHATMAN, Florham Park, N.J.), washing with 1 M sodium bicarbonate (pH 9.0), followed by an acetone wash. Counts were then determined by liquid scintillation (PERKINELMER, Wellesley, Mass.) . To control for the different specific activities of the preparations of SirT1, 2, and 3, the enzymes were first titrated in an NER using acetylated bovine serum albumin (BSA) as substrate. The amount of enzyme required to label equal amounts of NAD⁺ were normalized and described as exchange units. In vivo H1 acetylation was analyzed by treating 293 cells for 18 hours in media containing 5 mM nicotinamide. H1 was purified as described (Albig, et al. (1998) supra) and analyzed in the NER. H1 acetylation and deacetylation was also visualized by western blot using H1 α-AcK26 antibody in the presence of recombinant H1b as competitor (0.35 μg/ml). H1 used in the deacetylation assay was isolated by HA affinity purification, from 293F cells transfected with pcDNA4/TO H1b-HA and treated for 24 hours in 5 mM nicotinamide and 4 hours in media containing 5 mM TSA.

Immunofluorescence. HeLa, 293T, and HepG2 cells were stained with SirT1 antibodies and DAPI following standard immunofluorescence protocols and fixed with paraformaldehyde (Rice, et al. (2002) Genes Dev. 16:2225-2230). Images were visualized using a ZEISS inverted fluorescence microscope.

Transfections. All transfections were carried out in 293F cells using the indicated plasmids and POLYFECT (QIAGEN, Valencia, Calif.) following the protocol provided by the manufacturer. Nuclear and cytoplasmic fractions were isolated by a modified protocol (Dignam, et al. (1983) Methods Enzymol. 101:582-598). Fractions were analyzed by immunoprecipitation with the indicated antibodies followed by immunoblotting, or by luciferase activity (TD20e luminometer; Turner BioSystems, Sunnyvale, Calif.) . In the case of transient transfections, a β-gal reporter was used as a transfection control and the levels of luciferase activity were normalized to those of β-galactosidase, shown as a percentage. dsRNAi was ordered as a custom SMARTPOOL against SirT1 (Dharmacon, Lafayette, Colo.) and was transfected into U2OS cells using RNAFECT (QIAGEN, Valencia, Calif.). After incubation for 48 hours, cells were harvested and lysed with RIPA buffer (50 mM Tris-HCl, pH 7.4, 1% NP40, 0.25% Na-deoxycholate, 150 mM NaCl, 1 mM EDTA, 1 mM PMSF, 1 mM leupeptin, 1 mM aprotinin, and 1 mM pepstatin). Western blots were performed using the indicated antibodies. Total H4 was visualized by both western and COOMASSIE blue staining.

Plasmids. All constructs were generated using standard PCR-based cloning strategy, and the identities of individual clones were verified through DNA sequencing using an ABI PRISM DNA sequencer. Full-length SirT1, 2, and 3, as well as SirT1 truncations were cloned in Pet30c (NOVAGEN, Madison, Wis.) for expression in E. coli, FLAG-CMV4 (SIGMA, St. Louis, Mo.) for mammalian expression, the entire sequence including the FLAG tag from CMV4 into pAcHLT-B (BD BIOSCIENCES, San Diego, Calif.) for preparation of infectious baculovirus (Orbigen, San Diego, Calif.), and the entire sequence, including the FLAG tag and hexa-His tag from pAcHLT-B at the N terminus, into pcDNA4/TO (INVITROGEN, Carlsbad, Calif.) for inducible mammalian expression.. The QUICKCHANGE mutagenesis kit (STRATAGENE, La Jolla, Calif.) was used to generate a mutation in SirT1 that changes residue 363 from His to Tyr, designated H363Y. Sequence corresponding to Gal4 DBD (residues 1-147) was inserted into pcDNA4/TO for construction of inducible Gal4-fusion proteins. SirT1 was inserted in frame, C-terminal to the Gal4 DBD in pcDNA4/TOGal4DBD. Gal4-TK-Luc was used in transfection assays (Zhang, et al. (1998) Mol. Cell 1:1021-1031) and the neomycin resistance gene was inserted at the NdeI site for stable cell line selection. Human histone H1b and H1° were inserted into HA-Pet30c. As a β-Gal transfection control, the pCH110 Eukaryotic Assay vector was used (Pharmacia Biotech, Piscataway, N.J.).

Protein Expression and Purification. Recombinant SirT1 and SirT3 were expressed from baculovirus in SF9 cells and purified by M2 FLAG affinity chromatography washing with buffer C containing 500 mM KC1 and 0.01% NP40, followed by purification with Ni⁺-NTA resin under native conditions (QIAGEN, Valencia, Calif.). Recombinant SirT2 was expressed in E. coli strain BL21 (DE3) and was purified with Ni⁺-NTA resin under native conditions (QIAGEN, Valencia, Calif.). Recombinant histone H1 proteins were expressed in E. coli strain BL21 (DE3) and were purified with Ni+-NTA resin under denaturing conditions (QIAGEN, Valencia, Calif.) and adjusted to buffer C containing 100 mM KCl by serial dialysis. FLAG-tagged human p300 was expressed from baculovirus in SF9 cells and purified by standard M2 FLAG affinity purification. Gel filtration analysis of endogenous SirT1 in HeLa nuclear extract (Dignam, et al. (1983) Methods Enzymol. 101:582-598) was performed on a SUPEROSE 6 column (Pharmacia, Piscataway, N.J.) equilibrated with buffer C containing 500 mM KCl. Native mass was determined by comparison to the elution pattern of molecular weight markers. Native purification from HeLa nuclear extract (Dignam, et al. (1983) supra) was carried out as depicted in Scheme 1.

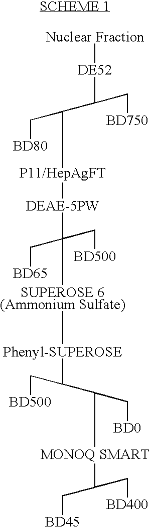

Buffer D (BD) contains 50 mM Tris-HCl [pH 7.9], 0.2 mM EDTA, 10% glycerol, 1 mM DTT, 0.2 mM PMSF, and the indicated mM concentration of ammonium sulfate. An ammonium sulfate precipitation was performed before SUPEROSE 6 gel filtration.

Identification of Interacting Proteins. SirT1 was purified from nuclear extract derived from a stable cell line expressing FLAG CMV4 SirT1. The nuclear extract was adjusted to buffer C containing 100 mM KCl and fractionated on a DE52 cellulose column. The presence of SirT1 was gauged using the NER along with western blot analysis using FLAG-specific antibodies, as well as SirT1-specific antibodies. Peak fractions were pooled, adjusted to buffer C containing 100 mM KCl, and purified by affinity purification using FLAG M2 resin followed by elution with FLAG peptide. This fraction was further affinity purified using a SirT1 monoclonal antibody resin and specific proteins were eluted using SirT1 peptide expressed in E. coli. Resins were washed as described in interaction assays. SirT1 peptide was then removed from the eluate by separation on a DE52 cellulose column. Proteins were visualized by COOMASSIE blue staining and bands were excised from the gel and identified. Gel-resolved proteins were digested with trypsin, peptide mixtures fractionated on a Poros 50 R2 RP micro-tip, and analyzed by matrix-assisted laser-desorption/ionization reflectron time-of-flight (MALDI-reTOF) MS using a BRUKER ULTRAFLEX TOF/TOF instrument (Bruker Daltonics, Billerica, Mass.), as described (Erdjument-Bromage, et al. (1998) J. Chromatogr. A. 826:167-181; Winkler, et al. (2002) Methods 26:260-269). Selected masses (m/z) were taken to search the human segment of the “NR” protein database (National Center for Biotechnology Information; Bethesda, Md.), utilizing the “PeptideSearch” algorithm (obtained from Matthias Mann, Southern Denmark University, Odense, Denmark). Mass spectrometric sequencing of selected peptides was done by MALDI-TOF/TOF (MS/MS) analysis on the same prepared samples, using the ULTRAFLEX instrument in “LIFT” mode. Fragment ion spectra were taken for database searches using the MASCOT MS/MS Ion Search program (Matrix Science Ltd., London, UK). Identifications were verified by comparing computer-generated fragment ion series of the predicted tryptic peptide with experimental MS/MS data.

Stable Cell Lines. 293F cells were transfected with plasmids FLAG CMV4 SirT1 and stable clones were selected by resistance to the drug G418 (Gemini Bio Products, West Sacramento, Calif.) . Gal4-SirT1 inducible cells were derived by transfection of pcDNA4/TO-Gal4-SirT1, and Gal4-TK-Luc-Neo into 293 TREX cells (INVITROGEN, Carlsbad, Calif.) and positive clones were selected for resistance to the drugs G418, Blasticidin (INVITROGEN, Carlsbad, Calif.), and Zeocin (INVITROGEN, Carlsbad, Calif.) . Cells were also selected for luciferase expression and Gal4-SirT1 repression and inducibility.

ChIP and RT-PCR. Standard ChIP protocols were performed (Kuo and Allis (1999) Methods 19:425-433) using Gal4-SirT1 inducible cells in the presence and absence of 1 mg/ml tetracycline. Before immunoprecipitation, both inputs (Tet−and Tet+) were normalized. The indicated antibodies were used at optimized conditions for each. Two different amounts of DNA eluted in the ChIP, 1x and 3x, were used in the PCRs for every set of primers. These experiments were repeated several times with consistent results.

Primers used in the PCR of the ChIP amplified region 1, between the TK promoter (Upper primer: 5′-GGC GAA TTC GAA CAC GCA GAT GC-3′, SEQ ID NO:4) and the beginning of the luciferase coding region (Lower primer (54-31): 5′-CTT CCA GCG GAT AGA ATG GCG CCG-3′, SEQ ID NO:5), region 2, (Upper primer (730-758): 5′-GCA CTG ATC ATG AAC TCC TCT GGA TCT AC-3′, SEQ ID NO:6; Lower primer (1031-1004): 5′-GAG AAT AGG GTT GGC ACC AGC AGC GCA C-3′, SEQ ID NO:7) and region 3, (Upper primer (1548-1567): 5′-CGA CGC AGG TGT CGC AGG TC-3′, SEQ ID NO:8; Lower primer (1719-1701): 5′-CAC AAC TCC TCC GCG CAA C-3′, SEQ ID NO:9). Reverse transcription PCR was performed from total RNA extracted from the indicated cell line using primers specific for luciferase region 2, GAPDH, (Upper primer (333-353): 5′-CGT CTT CAC CAT GGA GA-3′, SEQ ID NO:10; Lower primer (622-612): 5′-CGG CCA TCA CGC CCA CAG TTT-3′, SEQ ID NO:11), and the Neomycin resistance gene 3 Kb downstream of the luciferase gene (Upper primer (53-77): 5′-GGC TAT TCG GCT ATG ACT GGG CAC-3′, SEQ ID NO:12; Lower primer (327-302): 5′-GAG CAA GGT GAG ATG ACA GGA GAT CC-3′, SEQ ID NO:13).

### EXAMPLE 2

Human SirT1 Is a Nuclear Protein

To elucidate the function of human SirT1 protein in vivo, specific monoclonal antibodies were raised against the nonconserved regions of SirT1. A monoclonal antibody recognizing the C-terminus of human SirT1 was selected for further analyses. The antibody recognized the baculovirus-expressed recombinant FLAG-SirT1 protein and, importantly, a single protein of similar size present in nuclear fractions; no detection was apparent in cytoplasmic fractions. Importantly, the SirT1 monoclonal antibody failed to react with the other members of the Sirtuin family of proteins.

Immunofluorescence was subsequently performed to analyze the distribution of endogenous SirT1 protein in three commonly used but disparate cell types: HeLaS3, 293T, and HepG2. SirT1 localized in all cases to the nucleus, but the patterns of distribution were different: in HeLaS3 and 293T cells, SirT1 was present throughout the nuclei, in both euchromatin and heterochromatin regions. However, in some of the 293T cells, SirT1 seemed to be concentrated in certain regions, such as the inner nuclear envelope, certain DAPI-dense areas, and interestingly, in the nucleolus. Its presence in the nucleolus was consistent with other studies in which mouse SirT1 (mSir2α) appeared to regulate transcription by RNA polymerase I, in part, by deacetylation of TAF_{I}68 (Muth, et al. (2001) supra) . Another completely different pattern of SirT1 distribution was observed in HepG2 cells, in which the staining of SirT1 appeared concentrated in nuclear corpuscles that resembled PML bodies. The detection of SirT1 was specific, as the staining pattern was competed by recombinant SirT1 protein, but not by other SirT proteins.

### EXAMPLE 3

SirT1 is an NAD⁺-Dependent Histone Deacetylase

To characterize SirT1 activity in vitro, the ability of SirT1 to utilize acetylated substrates in the nicotinamide exchange reaction (NER) was analyzed. In this reaction an excess of [¹⁴C]-nicotinamide forces the reverse reaction, generating de novo-labeled NAD⁺(Landry, et al. (2000) supra) . SirT1-mediated generation of [¹⁴C]-NAD⁺was dependent on an acetylated substrate. Either acetylated histones or acetylated BSA fulfilled this requirement.

It was next demonstrated that SirT1 could deacetylate core histones in an NAD⁺-dependent manner using a TAU gel which separates histone polypeptides based on the extent of their acetylation. Incubation of SirT1 with human hyperacetylated histones resulted in deacetylation, and excess SirT1 was able to completely deacetylate all four core histones. Importantly, no activity was detected in the absence of NAD⁺. Unexpectedly, when limiting amounts of SirT1 were used in the reaction (0.1 nmol), SirT1 appeared to preferentially deacetylate histone H3 and H4; although at higher concentrations of SirT1, some deacetylation was observed in histone H2A. Since yeast silencing by Sir2p results in formation of repressive chromatin structure, which contains hypoacetylated H3 and H4, subsequent analysis concentrated on amino acid residues of these histones.

Two experiments were performed to identify the acetylated lysine residues of H3 and H4 that are preferentially targeted by SirT1. In both cases, SirT1 deacetylation activity was scored using western blot analysis and antibodies specific for different histone H3 and H4 acetylated residues. In the first case, increasing amounts of SirT1 were tested. In the second case, a time course experiment was performed with limiting amounts of SirT1. The results showed that SirT1 has a clearly detectable histone deacetylase activity with some preference for histone H4-K16.

Since an H4-K16 deacetylase has not been described for higher organisms, the level of acetylated H4-K16 upon reduction of the intracellular concentration of SirT1 was determined using RNAi. A collection of dsRNAs (SMARTPOOL) directed toward SirT1 were transfected into U20S cells. Cell extracts were analyzed for the presence of SirT1 and H4 acetylated residues K16, and K8 and H3 acetylated K9. Total histone H3 and H4 were also analyzed as were other histone “marks” known to be associated with repressive chromatin, e.g., trimethylated H3-K9 (Rea, et al. (2000) Nature 406:593-599) and monomethylated H4-K20 (Nishioka, et al. (2002) Mol. Cell 9:1201-1213). Decreased levels of SirT1 correlated with increased levels of H4-K16Ac, indicating that SirT1 is an H4-K16 deacetylase. There were no detectable changes in the levels of H4-K8 acetylation. Decreased amounts of SirT1 also generated increased amounts of acetylated H3-K9 and a concomitant decrease in methylated H3-K9. Similarly, decreased SirT1 resulted in a decrease of methylated H4-K20, a repressive mark. The observed differences were not due to different amounts of histones H3 and H4. These studies collectively indicate that SirT1 deacetylates histone polypeptides in vivo, resulting in the establishment of repressive chromatin marks.

### EXAMPLE 4

SirT1 is the Main Nuclear NAD⁺-Dependent Deacetylase In Vivo

Biochemical purification of endogenous SirT1 from human S3 nuclear fractions or HeLa cells rendered a single SirT1 polypeptide that fractionated with an apparent mass of ˜350 kDa. This result supports previous data with HsT2 that suggest that the Sir2 family members form trimers (Zhao, et al. (2003) Nat. Struct. Biol. 10:864-871).

Nuclear extracts from a stable cell line expressing FLAG-tagged SirT1 were fractionated on a DE52 cellulose column and SirT1 complexes were isolated using two consecutive affinity purification steps (α-FLAG and α-SirT1). The recovered proteins were separated by electrophoresis on SDS-PAGE gel followed by silver staining. Proteins were excised from the gel and identified by a combination of peptide mass fingerprinting using matrix-assisted laser desorption/ionization-time-of-flight (MALDI-TOF) mass spectrometry (MS), and mass spectrometric sequencing using MALDI-TOF/TOF MS/MS. Most of the polypeptides identified corresponded to SirT1 ; however, histone H1b (or H1.4) was also detected in substoichiometric amounts. Since H1b is an H1 isoform implicated in heterochromatin [D'Incalci, et al. (1986) Eur. J. Biochem. 154:273-279; Higurashi, et al. (1987) J. Biol. Chem. 262:13075-13080; Parseghian, et al. (2001) supra), the presence of H1b in the SirT1 preparation was indicative of a functional connection.

### EXAMPLE 5

The N-Terminus of SirT1 Directly Interacts with Histone H1

The validity of SirT1 and histone H1 interaction was analyzed. Extracts from 293 cells transfected with HA-tagged H1b were immunoprecipitated using anti-HA resin. Endogenous SirT1 coimmunoprecipitated with histone H1b even under the very stringent conditions used (500 mM KCl and 0.01% NP-40) . Subsequently, in vitro pull-down experiments were performed using purified recombinant HA-H1b and FLAG-SirT1 proteins. The results clearly demonstrated that SirT1 binds directly to H1b, as each antibody coimmunoprecipitated the candidate protein, dependent upon the presence of the antibody-specific protein.

The domain in SirT1 required for this interaction with H1b was mapped. A SirT1 mutant lacking the first 268 residues abolished the interaction. The deleted residues correspond to the unique SirT1 N-terminal sequence. A fusion protein containing the SirT1-specific N-terminal domain fused to FLAG (SirT1 1-268) was specifically immunoprecipitated with HA-H1b. It was also determined whether other H1 isoforms could interact with SirT1. This analysis indicated that H1° and H1d (H1.2) both interacted with the SirT1-specific N-terminal domain in vitro. However, ChIP experiments indicated that Gal4-SirT1 was able to recruit overexpressed HA-tagged H1b, but not overexpressed HA-H1°.

### EXAMPLE 6

Histone H1 is Deacetylated by SirT1

It has been shown that H1 is acetylated on serine 1 (Gurley, et al. (1995) J. Biol. Chem. 270:27653-27660); however, acetylation on H1-lysine residues has not been reported. Thus the following experiments were performed. Two different amounts (20 and 40 μg) of purified calf thymus H1 were either untreated (H1 ) or pretreated (H1-T1) with recombinant SirT1, in the presence of NAD⁺, and then tested as substrates in an NER reaction. The H1 proteins were purified by perchloric acid extraction, a procedure that separates H1 specifically from the other cellular proteins due to its high charge (Albig, et al. (1998) supra) . Subsequently, the perchloric-purified H1 proteins were used as substrate in the NER reaction in the presence or absence of SirT1. Histone H1 served as substrate in the SirT1-dependent NER assay, but preincubation of histone Hi with SirT1 drastically impaired NER activity. Importantly, the activity observed was specific, as western blot analysis using two different sources for H3 and H4 antibodies failed to detect H3 and H4 in the histone Hi preparation. H1 purified from nicotinamide-treated 293 cells rendered it a better substrate in the NER reaction relative to the nontreated case. Thus, histone H1 is acetylated in vivo and can be deacetylated by SirT1 in vitro. Since the NER assay is dependent on an acetylated substrate, and the pretreatment of histone H1 with SirT1 drastically decreased but did not abolish completely its ability to support NER activity, SirT1 may not deacetylate all acetylated H1 residues.

SirT1 activity on histone H1 was directly tested in vitro. H1 was acetylated in vitro by p300-HAT in the presence of [³H]-AcCoA and then incubated with different amounts of SirT1. SirT1 -released up to 70% of the total label in Hi, but a plateau was reached, and the reaction was not completed. This indicated that SirT1 may not be able to utilize all p300-acetylated residues as substrates. The same result was observed using recombinant human H1b; however, specificity was also scored in this assay using different SirT proteins. These results established that H1b deacetylation is specific for SirT1, as treatment with equal NAD⁺-exchange units (measured using acetylated BSA as substrate) of SirT2 or SirT3 failed to significantly deacetylate H1b.

The H1 residue(s) targeted for acetylation were subsequently identified. Several observations pointed to lysine 26. First, SirT1 is a component of an Ezh2-containing complex, PRC4, which specifically methylates H1-K26. Second, the sequence around H1-K26 and histone H3-K27 is conserved and H3-K27 is acetylated in yeast in vivo (Suka, et al. (2001) Mol. Cell 8:473-479). Finally, H1-K26 is highly conserved among the different isoforms of H1 and between species. Dot-blot analysis with an antibody generated against a peptide containing residues 21-31 of histone H1b, acetylated at K26, demonstrated its specificity. No immunodetection was observed with H4 N-terminal tail peptides (1-20) acetylated at K16 or K12, an unmodified H1b peptide (residues 21-31), or the same peptide trimethylated at K26. A modest immunoreactivity was observed with an H3 peptide acetylated at lysine 27. Antibody reactivity against H1 histones isolated from cells treated with TSA and nicotinamide (HDAC inhibitors) was compared with recombinant H1b expressed in E. coli. Only mammalian H1 was recognized by the antibody, establishing the existence of H1-Ac-K26 in vivo. Finally, treatment of native H1 with SirT1 followed by western blot analysis using the acetyl H1-K26 antibody resulted in decreased immunoreactivity, demonstrating that acetylated H1-K26 is a substrate for SirT1 in vitro.

### EXAMPLE 7

Targeting of SirT1 to a Promoter Results in Repression

It has been shown that SirT1 can be recruited to promoters in vivo by sequence-specific regulators (Fulco, et al. (2003) supra). Thus, to determine the in vivo connection between SirT1 and chromatin regulation, SirT1 was fused to the Gal4 DNA binding domain (G4BD) to target it to promoters containing Gal4 sites. Transient transfection assays were performed using a pTK-Luc reporter under the control of Gal4 and different fusion proteins such as G4BD alone, the activator G4-VP16, the repressors G4-MeCP2 and G4-SirT1, the catalytically inactive G4-SirT1 (H363Y) (Imai, et al. (2000) supra; Tanny and Moazed (2001) Proc. Natl. Acad. Sci. USA 98:415-420), and the deletion mutant G4-ΔNSirT1 (Δ(1-268)). The results of this analysis showed that G4-VP16 activated luciferase expression. Targeting of SirT1 to the reporter inhibited reporter expression to a level similar to that obtained with G4-meCP2, approximately 100%. Unexpectedly, G4-SirT1 (H363Y), containing a single substitution in the active site that abolished enzymatic activity, resulted in approximately 40% repression. This result indicated that part of the SirT1 repressive mechanism can be attributed to an interaction with other proteins, possibly H1, and not solely to the SirT1 enzymatic activity. Endogenous SirT1 forming mixed trimers with mutant SirT1 could not explain this effect, as the purification of transfected FLAG-SirT1 (H363Y) from cells using anti-FLAG agarose yielded a completely inactive protein. Consistent with this, the deletion of the N-terminal region of SirT1 (1-268), which mediates H1 interaction, resulted in derepression of ˜20% (compare G4-SirT1 with G4-ΔN-SirT1 ).

To analyze SirT1 function in the context of chromatin, a 293-cell line containing a stably integrated Gal4-SirT1 under the control of a Tet-on promoter was created. These cells also contained a stably integrated TK-luciferase reporter containing Gal4 binding sites and were selected for luciferase expression, likely due to the integration of the reporter into euchromatic regions. Upon treatment of the cells with tetracycline, Gal4-SirT1 was expressed, and luciferase expression was inhibited. This repression was specific to the Gal4-Luc reporter, as expression of the NEO reporter (3 kb away from the Luc promoter) or the endogenous GAPDH control was not affected by tetracycline. Using this- system, the recruitment of factors to the luciferase gene was analyzed as was their affect on specific histone modifications by ChIP analyses.

Upon induction, Gal4-SirT1 was specifically detected on the promoter of the TK-luciferase reporter. This resulted in the overall deacetylation of histones H3 and H4. Most importantly, the recruitment of endogenous histone H1 to the promoter and only to the promoter region was demonstrated, as H1, like SirT1, was not detected within the coding sequences. Even when histone H1b was overexpressed upon transfection, H1 was again detected only at the promoter of the reporter and only upon induction of SirT1 expression. The presence of SirT1 and histone H1 was accompanied by a loss of acetylation of H4-K16 that was also restricted to the promoter region.

Since acetylation/deacetylation and methylation of histones can be coordinately regulated (Berger (2002) supra; Shankaranarayana, et al. (2003) Curr. Biol. 13:1240-1246), it was determined whether SirT1 targeted to the TK-luciferase reporter also affected other histone modifications, an effect supported by the SirT1-RNAi experiments described herein. Repression of luciferase expression was accompanied by the appearance of methylated H4-K20, and also by an increase in methylation of K3-K9 (Peters, et al. (2002) Nat. Genet. 30:77-80). These two repressive marks, although concentrated within the promoter region, also extended to the coding sequence. No change was observed in methylation at H3-K4, a mark associated with open chromatin (Santos-Rosa, et al. (2002) Nature 419:407-411). This is consistent with observations demonstrating that methylation of K4 apparently does not change during repression of genes located within facultative heterochromatin in higher eukaryotes (Schneider, et al. (2004) Nat. Cell Biol. 6:73-77). Of special interest was the result pertaining to the H3-K79 methyl mark. This modification occurs in the globular domain of H3 and, in yeast, restricts the spreading of Sir2p and therefore silenced loci (Lacoste, et al. (2002) supra; Ng, et al. (2002) supra; van Leeuwen, et al. (2002) supra) . K79 was found to be hypomethylated in repressed regions in mammals (Ng, et al. (2003) Proc. Natl. Acad. Sci. USA 100:1820-1825). Thus, the methyl-H3-K79 mark functions as a “boundary” separating active and inactive chromatin domains. When ChIP analysis was performed using α-methyl K79 antibodies, the presence of SirT1 at the Luc promoter resulted in the reduction of K79 methylation that spanned the promoter and coding sequences.

What is claimed is:

1. A method for promoting formation of facultative heterochromatin comprising contacting a cell with human sirtuin type 1 thereby modulating the acetylation of histones and promoting formation of facultative heterochromatin.

2. An isolated antibody raised against a C-terminal fragment of human sirtuin type 1.

3. An isolated antibody raised against residues 21-31 of histone H1b, wherein lysine 26 of said histone H1b is acetylated.

4. A method for diagnosing a cancer comprising contacting a sample with the antibody of claim 2, detecting the level of human sirtuin type 1 protein in the sample and comparing said level with a control, wherein an elevated level of human sirtuin type 1 protein in the sample as compared to the control is indicative of cancer.

5. A kit comprising the antibody of claim 2.
